(19) United States
(12) Patent Application Publication
(10) Pub. No.: US 2026/0306117 A1
(43) Pub. Date: Oct. 8, 2026

(54) SYSTEMS, DEVICES, AND METHODS FOR TREATING VESSEL OCCLUSIONS

(71) Applicant: Covidien LP, Mansfield, MA (US)

(72) Inventor: Ramon T. CARRILLO, Santa Ana, CA (US)

(21) Appl. No.: 19/554,952

(22) Filed: Mar. 3, 2026

Related U.S. Application Data
(60) Provisional application No. 63/782,230, filed on Apr. 2, 2025.

(51) Int. Cl. A61B 17/22

(52) U.S. Cl. CPC A61B 17/22; A61B 2017/22038; A61B 2017/22079; A61B 2017/22082; A61B 2217/005

(57) ABSTRACT

Treatment devices and associated methods and systems are disclosed herein. According to some embodiments, an aspiration system includes a catheter having a proximal end, a distal end, and a lumen extending therebetween. The system further includes a vacuum source configured to provide vacuum to the lumen and an actuation mechanism coupled to the distal end of the catheter and configured to selectively extend the distal end in a distal direction by a controlled distance, such that extension of the distal end facilitates engagement and retrieval of a clot.

### CROSS-REFERENCE TO RELATED APPLICATIONS

This application claims the benefit of U.S. Provisional Patent Application Ser. No. 63/782,230, filed Apr. 2, 2025, the entire content of which is incorporated herein by reference.

### TECHNICAL FIELD

The present technology relates to systems and methods for removing obstructions from body lumens. Some embodiments of the present technology relate to aspiration catheters and associated components.

### BACKGROUND

Many medical procedures use medical device(s) to remove an obstruction (such as clotting material) from a body lumen, vessel, or other organ. An inherent risk in such procedures is that mobilizing or otherwise disturbing the obstruction can potentially create further harm if the obstruction or a fragment thereof dislodges from the retrieval device. If all or a portion of the obstruction breaks free from the device and flows downstream, it is highly likely that the free material will become trapped in smaller and more tortuous anatomy. In many cases, the physician will no longer be able to use the same retrieval device to again remove the obstruction because the device may be too large and/or immobile to move the device to the site of the new obstruction.

Procedures for treating ischemic stroke by restoring flow within the cerebral vasculature are subject to the above concerns. The brain relies on its arteries and veins to supply oxygenated blood from the heart and lungs and to remove carbon dioxide and cellular waste from brain tissue. Blockages that interfere with this blood supply eventually cause the brain tissue to stop functioning. If the disruption in blood occurs for a sufficient amount of time, the continued lack of nutrients and oxygen causes irreversible cell death. Accordingly, it is desirable to provide immediate medical treatment of an ischemic stroke.

To access the cerebral vasculature, a physician typically advances a catheter from a remote part of the body (typically a leg) through the abdominal vasculature and into the cerebral region of the vasculature. Once within the cerebral vasculature, the physician deploys a device for retrieval of the obstruction causing the blockage, for example an aspiration catheter. Concerns about dislodged obstructions or the migration of dislodged fragments increases the duration of the procedure at a time when restoration of blood flow is paramount. Furthermore, a physician might be unaware of one or more fragments that dislodge from the initial obstruction and cause blockage of smaller more distal vessels. Accordingly, there remains a need for improved devices and methods that can remove occlusions from body lumens and/or vessels.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1 is a side view of an example treatment system in accordance with examples of the present technology.

FIG. 2A-2D are side, enlarged side, top, and perspective end views, respectively, of a treatment device in accordance with examples of the present technology.

FIGS. 3A and 3B are perspective and top views, respectively, of a clot engagement region of a treatment device in accordance with examples of the present technology.

FIGS. 4A-4C are side, perspective cross-sectional, and enlarged perspective cross-sectional views, respectively, of a treatment device engaging a clot within a blood vessel in accordance with examples of the present technology.

### DETAILED DESCRIPTION

In various implementations, the present technology provides aspiration catheters with enhanced clot engagement and retrieval capabilities. The aspiration catheters include a flattened portion proximate to the distal end that defines a clot engagement surface with strategically positioned perforations for applying suction to thrombus material. In some implementations, the flattened configuration allows the catheter to maintain close wall apposition even in small diameter vessels while providing sufficient space for blood flow around the device. A flared tip positioned distal to the flattened portion prevents captured thrombus from sliding off during retrieval.

The aspiration catheters described herein may be particularly suitable for neurovascular applications where conventional cylindrical aspiration catheters can face challenges in maintaining effective clot engagement. The flattened profile enables the aspiration catheter to navigate tortuous vasculature while providing enhanced clot capture capabilities through the perforated sidewalls. Additionally or alternatively, the combination of the flattened portion and flared distal tip creates a mechanical advantage in clot retrieval, as the expanded tip diameter helps prevent the clot from escaping distally during withdrawal while the flattened section maintains consistent vacuum application through its perforations.

In some implementations, the aspiration catheters can be used in vessels having diameters as small as 2-4 mm, making them particularly well-suited for cerebral vessels. The flattened portion may be configured to maintain a low profile that minimizes vessel distension while still providing adequate flow lumen cross-sectional area for effective aspiration. The perforations in the sidewalls of the flattened portion may be sized and arranged to optimize clot engagement while preventing fragmentation that could lead to distal embolization.

Various other features and aspects of example treatment systems and devices are described in more detail below. The present technology provides systems, devices, and methods for treating various medical conditions, including removing a thrombus or other occlusion from a bodily lumen. Although many of the embodiments are described below with respect to devices, systems, and methods for treating a cerebral or intracranial embolism, other applications and other embodiments in addition to those described herein are within the scope of the technology. For example, the treatment systems and methods of the present technology may be used to remove emboli from body lumens other than blood vessels (e.g., the digestive tract, etc.) and/or may be used to remove emboli from blood vessels outside of the brain (e.g., pulmonary, abdominal, cervical, or thoracic blood vessels, or peripheral blood vessels including those within the legs or arms, etc.). In addition, the aspiration systems and methods of the present technology may be used to remove luminal obstructions other than clotting material (e.g., plaque, resected tissue, foreign material, etc.). Additionally or alternatively, the treatment systems and devices described herein can be used for fluid delivery to a treatment site within a bodily lumen (e.g., delivery of medicament, saline, contrast media, or other suitable fluid to an intravascular treatment site within a cerebral vessel or other desired treatment site).

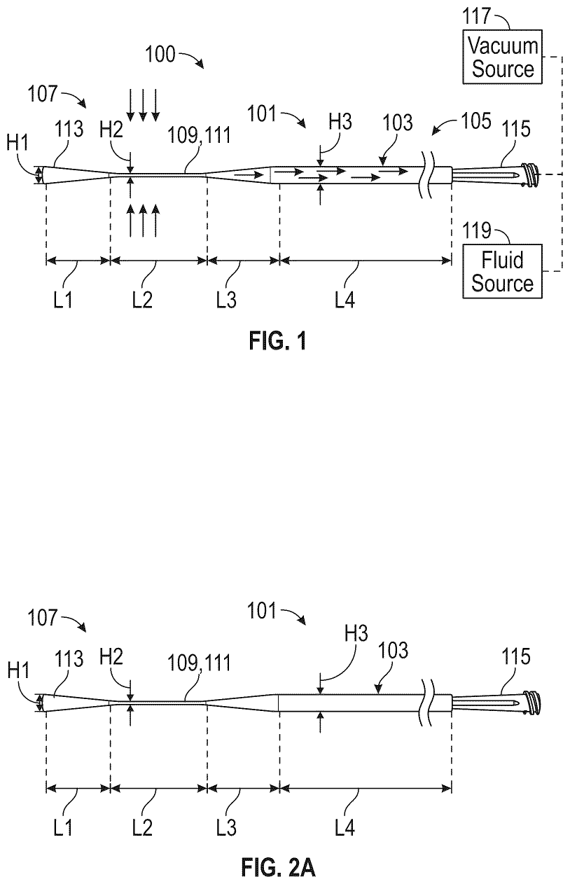

Referring to FIG. 1, a treatment system 100 is illustrated in accordance with examples of the present technology. The treatment system 100 includes a treatment device 101, which in some implementations can be configured as an aspiration catheter. In the implementation shown, the treatment device 101 includes an elongate shaft 103 extending between a proximal portion 105 and a distal region 107. The elongate shaft 103 may be formed from flexible polymeric materials suitable for navigating tortuous vasculature in various implementations. The distal region 107 includes a flattened portion 109 with a plurality of sidewall perforations 111. The sidewall perforations 111 are configured to apply suction to thrombotic material within a blood vessel when the treatment device 101 is deployed in vivo. In some implementations, the sidewall perforations 111 may be arranged in predetermined patterns along one or both sidewalls of the flattened portion 109 to optimize clot engagement.

A flared distal tip 113 is positioned distal to the flattened portion 109. The flared distal tip 113 has an expanded diameter relative to the flattened portion 109, which helps prevent captured thrombus from sliding off during withdrawal of the catheter. In some implementations, the flared distal tip 113 may have a gradually increasing diameter in the distal direction to create a mechanical advantage in holding captured clot material during retrieval operations.

The elongate shaft 103 defines a central lumen (not shown in FIG. 1) extending from the proximal portion 105 through the flattened portion 109 and flared distal tip 113. In some implementations, the central lumen may have varying cross-sectional shapes along its length, transitioning from a circular cross-section in the proximal portion 105 of the elongate shaft 103 to an oval or flattened cross-section in the flattened portion 109. The sidewall perforations 111 are in fluid communication with the central lumen to enable aspiration of thrombus material. In some implementations, the perforations 111 may be sized and shaped to prevent passage of thrombus fragments larger than a predetermined size while maintaining effective suction capabilities.

As illustrated in FIG. 1, the treatment system 100 may additionally include a connector hub 115 coupled to the proximal portion 105 of the elongate shaft 103. The connector hub 115 may comprise a tapered body having a distal portion and a proximal portion, wherein the distal portion is configured to engage with the proximal portion 105 of the elongate shaft 103. The connector hub 115 defines a lumen extending therethrough, the lumen being in fluid communication with the lumen of the elongate shaft 103 when the components are mated. In some implementations, the connector hub 115 may be formed from a polymeric material having sufficient rigidity to maintain structural integrity during medical procedures while providing adequate frictional engagement with the proximal portion 105.

A vacuum source 117 can be operably coupled to the treatment device 101 via the connector hub 115. In some implementations, the vacuum source 117 can be configured to generate negative pressure within the lumen to facilitate aspiration through the perforations 111. The vacuum source 117 can be adjustable to provide various levels of negative pressure suitable for different procedural requirements. In various implementations, the vacuum source 117 can take the form of a pump, syringe, or other suitable device configured to supply negative pressure to the lumen.

The treatment system 100 can further include a fluid source 119 operably coupled to the treatment device 101. In some implementations, the fluid source 119 can be configured to deliver fluids through the lumen to exit the device 101 via the perforations 111. The delivered fluids can include, for example, saline, contrast media, therapeutic agents, or combinations thereof. In various implementations, the fluid source 119 can take the form of a pump, syringe, or other suitable delivery mechanism coupled to or including a reservoir containing fluid to be delivered through the lumen of the elongate shaft 103.

Additionally, a control system (not shown) may be provided to regulate the operation of the vacuum source 117 and/or the fluid source 119. The control system may be configured to adjust aspiration parameters, such as pressure levels and timing, and fluid delivery parameters, such as flow rate and volume. In some implementations, the control system may be programmable to implement predetermined operational sequences suitable for specific treatment protocols or patient conditions.

For general intravascular applications, the elongate shaft 103 can have an outer diameter ranging from about 3 French (Fr) to about 24 Fr, and an inner diameter ranging from about 0.020 inches to about 0.200 inches, depending on the target vessel size and intended use. In implementations configured specifically for neurovascular applications, the elongate shaft 103 can have a smaller profile with an outer diameter ranging from about 3 Fr to about 8 Fr at its distal end, corresponding to approximately 1.0 mm to 2.7 mm, and an inner diameter ranging from about 0.020 inches to about 0.088 inches to enable navigation through the tortuous vessels of the neurovasculature while maintaining sufficient lumen size for clot aspiration. Additionally or alternatively, the elongate shaft 103 can have a tapered configuration with a larger diameter at its proximal end transitioning to a smaller diameter at its distal end to balance trackability with aspiration efficiency.

FIG. 1 also illustrates example dimensional relationships between various portions of the treatment device 101. In some implementations, the treatment device 101 may comprise a first length L1 corresponding to the flared distal tip 113, a second length L2 corresponding to the flattened portion 109 with perforations 111, a third length L3 corresponding to a transition region between the flattened portion 109 and the standard cylindrical portion of the elongate shaft 103, and a fourth length L4 corresponding to the main body of the elongate shaft 103. The heights of different portions of the treatment device 101 may also vary, with a first height H1, second height H2, and third height H3 corresponding to the flared distal tip 113, the flattened portion 109, and the standard cylindrical portion of the elongate shaft 103, respectively.

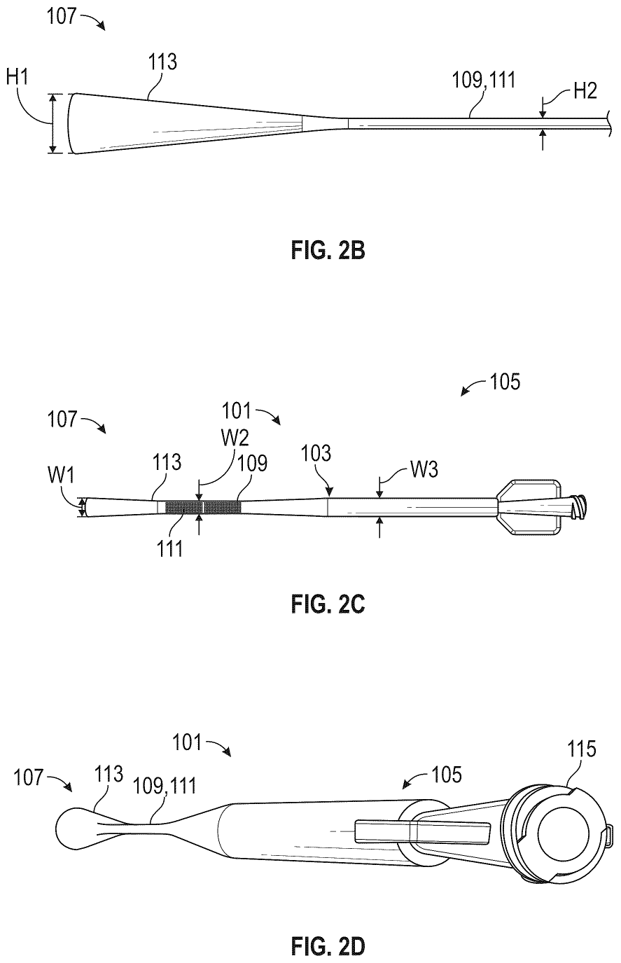

Referring now to FIGS. 2A-2D, various views of an exemplary treatment device 101 are shown to more clearly illustrate the structural features and dimensional relationships of the device components. FIG. 2A illustrates a side view of the treatment device 101. As shown in this view, the treatment device 101 includes the elongate shaft 103, the proximal portion 105, the distal region 107, the flattened portion 109 with perforations 111, the flared distal tip 113, and the connector hub 115. The elongate shaft 103 extends for the length LA, the transition region extends for the length L3, the flattened portion 109 extends for the length L2, and the flared distal tip 113 extends for the length L1. The heights of the respective portions are shown as H1 for the maximum height of the flared distal tip 113, H2 for the height of the flattened portion 109, and H3 for the height of the standard cylindrical portion of the elongate shaft 103.

In some implementations, the length L1 of the flared distal tip 113 may range from about 1 mm to about 10 mm, the length L2 of the flattened portion 109 may range from about 5 mm to about 50 mm, the length L3 of the transition region may range from about 2 mm to about 20 mm, and the length L4 of the elongate shaft 103 may range from about 50 cm to about 200 cm, depending on the intended application and target vessel location.

FIG. 2B provides an enlarged detail view of the distal region 107 of the treatment device 101. This view more clearly shows the flared distal tip 113 with height H1 and the flattened portion 109 with height H2. The perforations 111 are visible along the flattened portion 109. In some implementations, the flared distal tip 113 may have a maximum height H1 that is between about 1.2 and 3.0 times the height H2 of the flattened portion 109. The transition from the flattened portion 109 to the flared distal tip 113 may be gradual to facilitate smooth tracking through vasculature and to minimize trauma to vessel walls during advancement and retraction of the device.

FIG. 2C provides a top plan view of the treatment device 101, showing the elongate shaft 103, the proximal portion 105, the distal region 107, the flattened portion 109 with perforations 111, the flared distal tip 113, and the connector hub 115. From this perspective, the widths of the respective portions can be observed, with a first width W1 corresponding to the maximum width of the flared distal tip 113, a second width W2 corresponding to the width of the flattened portion 109, and a third width W3 corresponding to the width of the standard cylindrical portion of the elongate shaft 103. In some implementations, the width W2 of the flattened portion 109 may be between about 1.2 and 2.5 times the width W3 of the standard cylindrical portion of the elongate shaft 103. The width W1 of the flared distal tip 113 may be between about 1.2 and 2.0 times the width W2 of the flattened portion 109.

FIG. 2D is a rear perspective view of the treatment device 101, showing the connector hub 115 coupled to the proximal portion 105 of the elongate shaft 103. This view provides visualization of the internal lumen of the connector hub 115, which is in fluid communication with the lumen of the elongate shaft 103. The connector hub 115 may include standard luer fittings or other suitable connection mechanisms for secure attachment to the vacuum source 117 and/or fluid source 119 (shown in FIG. 1).

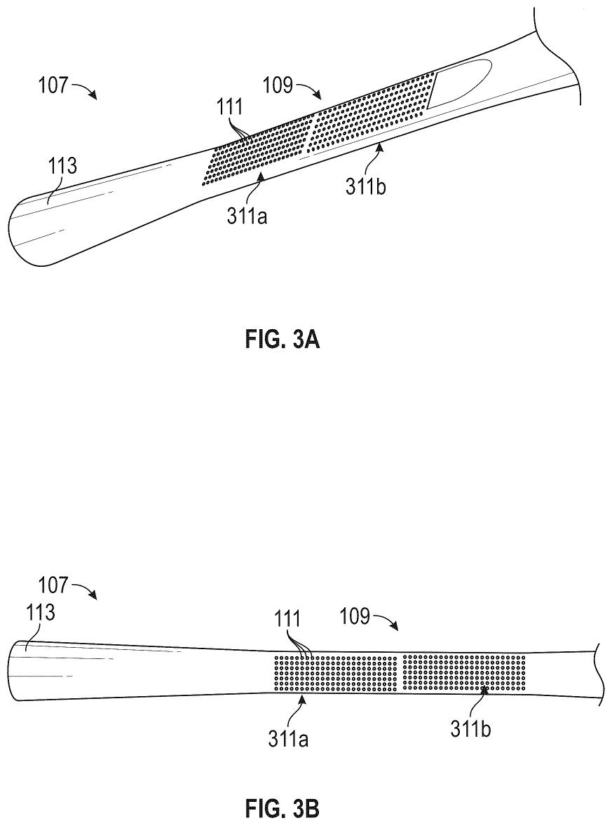

Referring now to FIGS. 3A and 3B, detailed views of the flattened portion 109 and perforations 111 of the treatment device 101 are shown. FIG. 3A provides a perspective view of the distal region 107 of the treatment device 101, showing the flared distal tip 113, the flattened portion 109, and the perforations 111. As seen in this view, the flattened portion 109 includes first and second perforation patterns 311a and 311b arranged on opposing sidewalls of the flattened portion 109. In some implementations, the perforation patterns 311a and 311b may comprise multiple rows of openings arranged to optimize thrombus engagement while maintaining structural integrity of the catheter walls. The perforation patterns 311a and 311b may be identical or may differ from each other in terms of perforation size, shape, spacing, or arrangement, depending on specific design considerations, for instance with distal perforation patterns 311a having larger individual openings, more individual openings, or otherwise a larger opening fraction than proximal perforation patterns 311b, or vice versa.

FIG. 3B illustrates a top view of the distal region 107 of the treatment device 101. This view more clearly shows the arrangement of the perforation patterns 311a and 311b along the flattened portion 109. In some implementations, the perforations 111 may have diameters ranging from about 0.2 mm to about 2.0 mm and may be spaced apart by distances ranging from about 0.5 mm to about 5.0 mm. The perforations 111 may be arranged in regular patterns, such as linear arrays, staggered arrays, or other configurations designed to maximize the effective aspiration area while preserving the structural integrity of the flattened portion 109.

The flattened configuration of the portion 109 creates a reduced profile in one dimension while maintaining or increasing the profile in the perpendicular dimension. This configuration allows the treatment device 101 to maintain close wall apposition along one side of a vessel while providing space for blood flow through the remaining portion of the vessel lumen. The flattened portion 109 thus enables the device to be positioned adjacent to a thrombus without completely occluding flow in the vessel, which can be particularly advantageous in neurovascular applications where maintaining some level of perfusion during the procedure may be beneficial.

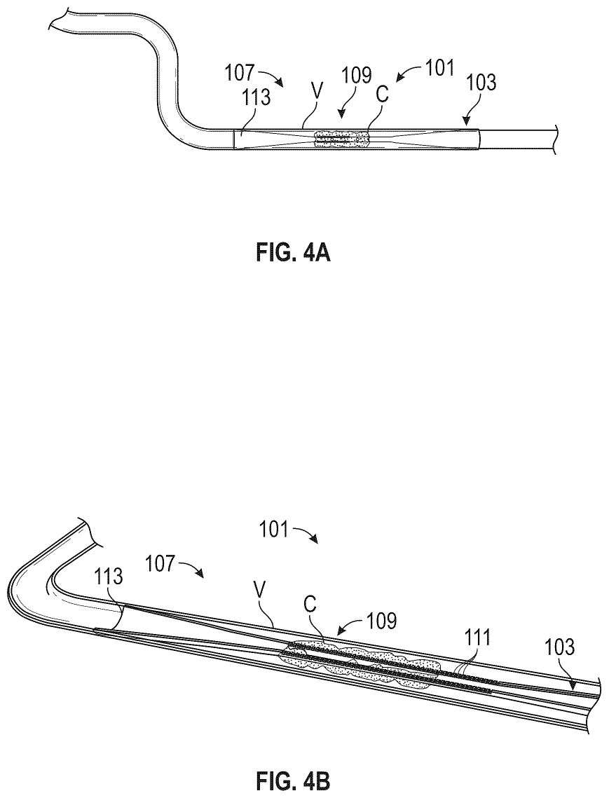

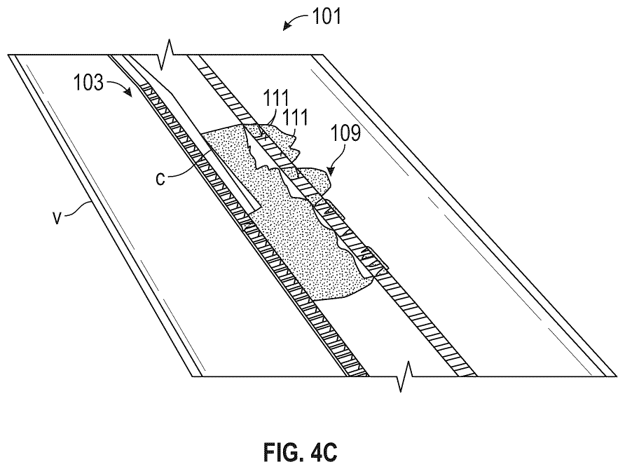

Referring now to FIGS. 4A-4C, various views of the treatment device 101 deployed within a blood vessel V for engagement and retrieval of a clot C are shown. FIG. 4A illustrates a side view of the treatment device 101 positioned within a blood vessel V, with the flattened portion 109 and perforations 111 oriented toward a clot C. The flared distal tip 113 has been advanced past the clot C, positioning the clot C adjacent to the flattened portion 109 and perforations 111. The elongate shaft 103 extends proximally through the vessel V. In the configuration shown in FIG. 4A, the treatment device 101 is positioned such that the flared distal tip 113 maintains apposition with the vessel wall.

FIG. 4B shows another view of the treatment device 101 within the blood vessel V, with the flattened portion 109 and perforations 111 engaging the clot C. In this view, suction is being applied through the perforations 111, drawing the clot C against the flattened portion 109. The flared distal tip 113 is positioned distal to the clot C, preventing the clot C from slipping distally during subsequent retrieval operations. The elongate shaft 103 extends proximally through the vessel V.

The configuration shown in FIG. 4B demonstrates how the flattened portion 109 and perforations 111 can effectively engage a clot C while the flared distal tip 113 prevents distal migration of the clot material. The application of suction through the perforations 111 helps secure the clot C against the treatment device 101, facilitating subsequent retrieval.

FIG. 4C provides a cross-sectional view of the treatment device 101 within the blood vessel V, showing the engagement between the perforations 111 and the clot C. In this view, the flattened configuration of the portion 109 can be observed in relation to the vessel lumen, illustrating how the device maintains wall apposition while preserving some flow space. The perforations 111 are shown applying suction to the clot C, drawing it against the flattened portion 109.

As shown in FIG. 4C, the clot C is securely engaged by the perforations 111, with the suction force distributed across multiple perforations to provide stable capture of the thrombotic material. The flattened configuration of the portion 109 provides a broader engagement surface compared to conventional cylindrical aspiration catheters, potentially improving the efficiency and reliability of clot capture.

In operation, the treatment device 101 may be deployed to a treatment site using conventional endovascular techniques. For example, the treatment device 101 may be advanced through a guide catheter or introducer sheath positioned in a vascular access site, such as a femoral artery. The treatment device 101 may be tracked over a guidewire to navigate tortuous vasculature and reach the target treatment site. Once positioned at the treatment site, the guidewire may be removed to allow for aspiration through the lumen of the elongate shaft 103.

With the treatment device 101 positioned at the treatment site, the flattened portion 109 may be oriented such that the perforations 111 face the clot C to be removed. This orientation may be achieved through rotational manipulation of the elongate shaft 103 from its proximal end. Once properly oriented, the flared distal tip 113 may be advanced past the clot C, positioning the clot C adjacent to the perforations 111 of the flattened portion 109.

With the clot C positioned adjacent to the perforations 111, suction may be applied through the lumen of the elongate shaft 103 via the vacuum source 117 (shown in FIG. 1). The applied suction creates negative pressure at the perforations 111, drawing the clot C against the flattened portion 109 and initiating engagement between the clot C and the treatment device 101. The distributed suction force applied through multiple perforations 111 helps secure the clot C against the treatment device 101 while minimizing the risk of clot fragmentation.

Once the clot C is securely engaged by the perforations 111, the treatment device 101 may be withdrawn from the vessel V to retrieve the clot C. During withdrawal, the flared distal tip 113 helps prevent the clot C from sliding off the distal end of the device, improving the reliability of clot retrieval. The flattened portion 109 maintains engagement with the clot C throughout the withdrawal process, with the applied suction helping to retain the clot material against the perforations 111.

In some implementations, fluid delivery through the lumen of the elongate shaft 103 may be employed in conjunction with aspiration to enhance clot engagement and retrieval. For example, a fluid such as saline or a thrombolytic agent may be delivered through the lumen and perforations 111 before, during, or after aspiration to help disrupt the clot structure or prevent vacuum lock against the vessel wall. The fluid delivery may be controlled by the fluid source 119 (shown in FIG. 1) and may be coordinated with the aspiration operation to optimize clot engagement and retrieval.

The treatment system 100 may be particularly suitable for removing clots or other obstructions from neurovascular structures, where conventional cylindrical aspiration catheters may face challenges in maintaining effective clot engagement while navigating tortuous anatomy. The flattened profile of the portion 109 enables the treatment device 101 to navigate tortuous vasculature while providing enhanced clot capture capabilities through the perforated sidewalls. Additionally or alternatively, the combination of the flattened portion 109 and flared distal tip 113 creates a mechanical advantage in clot retrieval, as the expanded tip diameter helps prevent the clot from escaping distally during withdrawal while the flattened section maintains consistent vacuum application through its perforations.

The described implementations of the treatment system 100 may be particularly advantageous in several respects. The flattened portion 109 with perforations 111 provides increased surface area for clot engagement compared to conventional cylindrical aspiration catheters, potentially improving the efficiency and reliability of clot capture. The flared distal tip 113 helps prevent captured clot material from sliding off during withdrawal, addressing a common challenge in aspiration thrombectomy procedures. The combination of these features may result in improved first-pass efficacy and reduced procedural time, which can be particularly important in time-sensitive conditions such as ischemic stroke.

Moreover, the flattened configuration of the portion 109 allows the treatment device 101 to maintain close wall apposition while preserving some flow space within the vessel lumen. This design feature may be beneficial in neurovascular applications, where maintaining some level of perfusion during the procedure can help preserve tissue function in the affected region. The ability to apply suction through multiple perforations 111 distributed across the flattened portion 109 may also help prevent vacuum lock against the vessel wall, a common issue with conventional end-hole aspiration catheters.

In various implementations, the treatment device 101 may be constructed from flexible polymeric materials suitable for endovascular use. For example, the elongate shaft 103 may comprise one or more layers of polyamide, polyethylene, polyurethane, polyester, silicone, or other suitable polymers. The shaft may include reinforcement structures, such as metal braids or coils, to provide torsional stability and kink resistance while maintaining appropriate flexibility for navigation through tortuous vasculature.

The flattened portion 109 may be formed by thermoforming, compression molding, or other suitable processes applied to the distal region 107 of the elongate shaft 103. The perforations 111 may be created by laser cutting, mechanical punching, or other precision manufacturing techniques. The flared distal tip 113 may be formed by heat shaping, molding, or attachment of a separate component to the distal end of the flattened portion 109.

In some implementations, the treatment device 101 may include radiopaque markers positioned at suitable locations, such as at the proximal and distal ends of the flattened portion 109 or at the flared distal tip 113. These markers may comprise bands, dots, or other configurations of radiopaque materials, such as gold, platinum, tantalum, or tungsten, to facilitate visualization of the device under fluoroscopy during deployment and operation.

Additionally or alternatively, the treatment device 101 may include a lubricious coating applied to its outer surface to reduce friction during navigation through vasculature. Suitable coating materials may include hydrophilic polymers, such as polyvinylpyrrolidone, or hydrophobic materials, such as polytetrafluoroethylene or silicone.

While the implementations described herein focus on aspiration thrombectomy applications, the treatment device 101 may also be adapted for other endovascular procedures. For example, the device may be used for controlled delivery of therapeutic agents to specific vascular sites, with the flattened portion 109 and perforations 111 providing targeted distribution of the agents. The device may also be used for diagnostic purposes, such as pressure monitoring or fluid sampling, with the flattened configuration allowing for these functions to be performed while maintaining some flow through the vessel.

Furthermore, the treatment system 100 may be modified for use in non-vascular applications, such as removal of obstructions from the biliary, urinary, or gastrointestinal tracts. In such applications, the dimensions and mechanical properties of the treatment device 101 may be adjusted to suit the specific anatomical and procedural requirements.

In additional implementations, the treatment device 101 may be integrated with sensing capabilities to provide real-time feedback during procedures. For example, pressure sensors may be incorporated near the distal region 107 to monitor pressure gradients across an obstruction, or flow sensors may be included to assess changes in flow patterns during aspiration. Such sensing capabilities may help optimize procedural parameters and improve outcomes.

Moreover, the treatment system 100 may be designed for compatibility with advanced imaging modalities, such as intravascular ultrasound (IVUS) or optical coherence tomography (OCT). In such implementations, the flattened portion 109 may include provisions for housing or interfacing with imaging elements, allowing for simultaneous visualization and treatment of vascular pathologies.

### EXAMPLES

The subject technology is illustrated, for example, according to various aspects described below, including with reference to FIGS. 1-4C. Various examples of aspects of the subject technology are described as numbered examples (1, 2, 3, etc.) for convenience. These are provided as examples and do not limit the subject technology.

Example 1. An aspiration catheter comprising: an elongate shaft having a proximal portion and a distal portion; a clot engagement section of the elongate shaft having a flattened configuration defining at least one sidewall, wherein the at least one sidewall includes one or more perforations configured to apply suction to a clot; and a flared tip disposed distal to the clot engagement section, wherein the flared tip has a diameter greater than an adjacent portion of the clot engagement section.

Example 2. The aspiration catheter of Example 1, wherein the one or more perforations are arranged in a linear pattern along the at least one sidewall.

Example 3. The aspiration catheter of any one of Examples 1-2, wherein the clot engagement section has a first height and a first width, and wherein the first width is greater than the first height.

Example 4. The aspiration catheter of any one of Examples 1-3, wherein the flared tip comprises a gradually expanding diameter in a distal direction.

Example 5. The aspiration catheter of any one of Examples 1-4, further comprising a vacuum source coupled to the proximal portion.

Example 6. The aspiration catheter of any one of Examples 1-5, wherein the elongate shaft defines a central lumen extending from the proximal portion to the distal portion.

Example 7. The aspiration catheter of any one of Examples 1-6, wherein the flattened configuration extends along a length between 1 cm and 5 cm.

Example 8. The aspiration catheter of any one of Examples 1-7, wherein the one or more perforations comprise a plurality of openings having a diameter between 0.2 mm and 2.0 mm.

Example 9. The aspiration catheter of any one of Examples 1-8, wherein the flared tip comprises a maximum diameter between 1.2 and 5.0 times a diameter of the clot engagement section.

Example 10. The aspiration catheter of any one of Examples 1-9, wherein the at least one sidewall comprises opposing sidewalls each including one or more perforations.

Example 11. The aspiration catheter of any one of the preceding Examples, wherein the clot engagement section comprises a material having greater flexibility than a material of the proximal portion of the elongate shaft.

Example 12. The aspiration catheter of any one of the preceding Examples, wherein the one or more perforations are arranged in a staggered pattern along the at least one sidewall.

Example 13. The aspiration catheter of any one of the preceding Examples, wherein the elongate shaft includes one or more radiopaque markers positioned adjacent to at least one of the clot engagement section and the flared tip.

Example 14. The aspiration catheter of any one of the preceding Examples, wherein the flared tip includes a plurality of circumferential slots configured to increase flexibility while maintaining the diameter of the flared tip.

Example 15. The aspiration catheter of any one of the preceding Examples, wherein at least a portion of the at least one sidewall includes a hydrophilic coating to reduce friction during advancement through a blood vessel.

Example 16. An aspiration device comprising: a tubular member extending between a proximal end and a distal end, wherein the proximal end is configured to be fluidically coupled to a vacuum source; a flattened region positioned proximate to the distal end, the flattened region having a first height less than a second height of an adjacent portion of the tubular member; and one or more aspiration openings formed in the flattened region, wherein a distal tip of the tubular member includes an expanded portion having a dimension greater than a corresponding dimension of the flattened region.

Example 17. The aspiration device of Example 16, wherein the flattened region comprises a first sidewall and a second sidewall, each sidewall including at least one aspiration opening.

Example 18. The aspiration device of any one of Examples 16-17, wherein the expanded portion comprises a gradual taper increasing in diameter toward the distal end.

Example 19. The aspiration device of any one of Examples 16-18, further comprising a fluid source coupled to the proximal end.

Example 20. The aspiration device of any one of Examples 16-19, wherein the one or more aspiration openings are arranged in a predetermined pattern.

Example 21. The aspiration device of any one of Examples 16-20, wherein the tubular member comprises a flexibility transition between the flattened region and the expanded portion.

Example 22. The aspiration device of any one of Examples 16-21, wherein the flattened region defines an oval cross-sectional shape.

Example 23. The aspiration device of any one of Examples 16-22, wherein the one or more aspiration openings are sized to prevent passage of thrombus material larger than a predetermined size.

Example 24. The aspiration device of any one of Examples 16-23, wherein the expanded portion comprises a maximum cross-sectional dimension between 20% and 400% greater than a corresponding dimension of the flattened region.

Example 25. The aspiration device of any one of Examples 16-24, wherein the flattened region maintains a consistent cross-sectional shape along its length.

Example 26. A method of removing thrombus from a blood vessel, comprising: advancing an aspiration catheter to a treatment site within the blood vessel, the aspiration catheter including a flattened portion having one or more perforations and a flared distal tip; positioning the flattened portion adjacent to a thrombus; applying suction through the one or more perforations to engage the thrombus against the flattened portion; and withdrawing the aspiration catheter while maintaining engagement between the thrombus and the flattened portion, wherein the flared distal tip inhibits the thrombus from sliding distally during withdrawal.

Example 27. The method of Example 26, further comprising aligning the flattened portion substantially parallel to a vessel wall at the treatment site.

Example 28. The method of any one of Examples 26-27, wherein applying suction comprises activating a vacuum source coupled to a proximal end of the aspiration catheter.

Example 29. The method of any one of Examples 26-28, further comprising injecting fluid through the aspiration catheter while applying suction.

Example 30. The method of any one of Examples 26-29, wherein positioning the flattened portion comprises rotating the aspiration catheter to orient the one or more perforations toward the thrombus.

Example 31. The method of any one of Examples 26-30, wherein withdrawing the aspiration catheter comprises maintaining the flattened portion substantially parallel to a longitudinal axis of the blood vessel.

Example 32. The method of any one of Examples 26-31, further comprising confirming engagement of the thrombus prior to withdrawing the aspiration catheter.

Example 33. The method of any one of Examples 26-32, wherein advancing the aspiration catheter comprises tracking the aspiration catheter over a guidewire.

Example 34. The method of any one of Examples 26-33, further comprising selecting an aspiration catheter size based on a diameter of the blood vessel at the treatment site.

### Conclusion

Although many of the embodiments are described above with respect to systems, devices, and methods for treating vessel occlusions in the brain, the technology is applicable to other applications and/or other approaches, such as vessel occlusions elsewhere in the body. As noted herein, in some implementations the treatment devices and systems disclosed herein can be used for delivery of fluid instead of or in addition to aspiration. This can include, for instance, delivery of fluid containing medicament (e.g., any substance used for medical treatment, diagnosis, disease prevention, and/or health promotion). Additionally, catheters as described herein can have applications for access purposes only. For instance, an extendable distal section can be manipulated independently of the proximal section, allowing for direct navigation to a lesion without the need for an additional guidewire. Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1-4.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

As used herein, the terms “generally,” “substantially,” “about,” and similar terms are used as terms of approximation and not as terms of degree, and are intended to account for the inherent variations in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

1. An aspiration catheter comprising:
an elongate shaft having a proximal portion and a distal portion;
a clot engagement section of the elongate shaft having a flattened configuration defining at least one sidewall, wherein the at least one sidewall includes one or more perforations configured to apply suction to a clot; and
a flared tip disposed distal to the clot engagement section, wherein the flared tip has a diameter greater than an adjacent portion of the clot engagement section.

2. The aspiration catheter of claim 1, wherein the one or more perforations are arranged in a linear pattern along the at least one sidewall.

3. The aspiration catheter of claim 1, wherein the clot engagement section has a first height and a first width, and wherein the first width is greater than the first height.

4. The aspiration catheter of claim 1, wherein the flared tip comprises a gradually expanding diameter in a distal direction.

5. The aspiration catheter of claim 1, further comprising a vacuum source coupled to the proximal portion.

6. The aspiration catheter of claim 1, wherein the elongate shaft defines a central lumen extending from the proximal portion to the distal portion.

7. The aspiration catheter of claim 1, wherein the flattened configuration extends along a length between 1 cm and 5 cm.

8. The aspiration catheter of claim 1, wherein the one or more perforations comprise a plurality of openings having a diameter between 0.2 mm and 2.0 mm.

9. The aspiration catheter of claim 1, wherein the flared tip comprises a maximum diameter between 1.2 and 5.0 times a diameter of the clot engagement section.

10. The aspiration catheter of claim 1, wherein the at least one sidewall comprises opposing sidewalls each including one or more perforations.

11. An aspiration device comprising:
a tubular member extending between a proximal end and a distal end, wherein the proximal end is configured to be fluidically coupled to a vacuum source;
a flattened region positioned proximate to the distal end, the flattened region having a first height less than a second height of an adjacent portion of the tubular member; and
one or more aspiration openings formed in the flattened region,
wherein a distal tip of the tubular member includes an expanded portion having a dimension greater than a corresponding dimension of the flattened region.

12. The aspiration device of claim 11, wherein the flattened region comprises a first sidewall and a second sidewall, each sidewall including at least one aspiration opening.

13. The aspiration device of claim 11, wherein the expanded portion comprises a gradual taper increasing in diameter toward the distal end.

14. The aspiration device of claim 11, further comprising a fluid source coupled to the proximal end.

15. The aspiration device of claim 11, wherein the one or more aspiration openings are arranged in a predetermined pattern.

16. The aspiration device of claim 11, wherein the tubular member comprises a flexibility transition between the flattened region and the expanded portion.

17. The aspiration device of claim 11, wherein the flattened region defines an oval cross-sectional shape.

18. The aspiration device of claim 11, wherein the one or more aspiration openings are sized to prevent passage of thrombus material larger than a predetermined size.

19. The aspiration device of claim 11, wherein the expanded portion comprises a maximum cross-sectional dimension between 20% and 400% greater than a corresponding dimension of the flattened region.

20. The aspiration device of claim 11, wherein the flattened region maintains a consistent cross-sectional shape along its length.

21. A method of removing thrombus from a blood vessel, comprising:
advancing an aspiration catheter to a treatment site within the blood vessel, the aspiration catheter including a flattened portion having one or more perforations and a flared distal tip;
positioning the flattened portion adjacent to a thrombus;
applying suction through the one or more perforations to engage the thrombus against the flattened portion; and
withdrawing the aspiration catheter while maintaining engagement between the thrombus and the flattened portion, wherein the flared distal tip inhibits the thrombus from sliding distally during withdrawal.

22. The method of claim 21, further comprising aligning the flattened portion substantially parallel to a vessel wall at the treatment site.

23. The method of claim 21, wherein applying suction comprises activating a vacuum source coupled to a proximal end of the aspiration catheter.

24. The method of claim 21, further comprising injecting fluid through the aspiration catheter while applying suction.

25. The method of claim 21, wherein positioning the flattened portion comprises rotating the aspiration catheter to orient the one or more perforations toward the thrombus.

26. The method of claim 21, wherein withdrawing the aspiration catheter comprises maintaining the flattened portion substantially parallel to a longitudinal axis of the blood vessel.

27. The method of claim 21, further comprising confirming engagement of the thrombus prior to withdrawing the aspiration catheter.

28. The method of claim 21, wherein advancing the aspiration catheter comprises tracking the aspiration catheter over a guidewire.

29. The method of claim 21, further comprising selecting an aspiration catheter size based on a diameter of the blood vessel at the treatment site.
